(19) United States
(12) Patent Application Publication
(10) Pub. No.: US 2016/0205904 A1
(43) Pub. Date: Jul. 21, 2016

(54) HUMAN-INDUCED PLURIPOTENT STEM CELLS, AND METHOD FOR PREPARING ANIMAL IN WHICH HUMAN IMMUNE SYSTEM IS EXPRESSED, BY USING SAME

(71) Applicant: CATHOLIC UNIVERSITY INDUSTRY-ACADEMIC COOPERATION FOUNDATION, Seoul (KR)

(72) Inventors: Ji Hyeon JU, Anyang-si (KR); Young Kyun KIM, Seoul (KR); Hyo Ju YI, Seoul (KR); Ju Ryun KIM, Incheon (KR); Hye Rin JEONG, Suwon-si (KR); Yeri A. RIM, Seoul (KR); Na rae PARK, Seoul (KR); Sejin YU, Anyang-si (KR); Seung Min JUNG, Seoul (KR)

(73) Assignee: CATHOLIC UNIVERSITY INDUSTRY-ACADEMIC COOPERATION FOUNDATION, Seoul (KR)

(21) Appl. No.: 14/917,246

(22) PCT Filed: Sep. 4, 2014
(86) PCT No.: PCT/KR2014/008324

§ 371 Date: Mar. 7, 2016

(30) Foreign Application Priority Data
Sep. 6, 2013 (KR) 10-2013-0107294

(51) Int. Cl. A01K 67/027

(52) U.S. Cl. CPC A01K 67/0278; A01K 2207/15; A01K 2227/105; A01K 2267/0387

(57) ABSTRACT

The present disclosure relates to: a method for preparing an animal in which the human immune system is expressed, by using human-induced pluripotent stem cells; and an animal prepared by the method.

### TECHNICAL FIELD

The present invention relates to a method of preparing an animal in which a human immune system is expressed using human-induced pluripotent stem cells (human-iPS cells), and an animal produced by the same method.

### BACKGROUND TECHNOLOGY

Induced pluripotent stem cells (iPS cells) refer to cells with pluripotency, obtained by dedifferentiating from differentiated cells such as somatic cells, and enabling to differentiate into various organ cells. Since iPS cells may be obtained by dedifferentiating (reprogramming) differentiated cells by dedifferentiation (reprogramming) inducers, a patient immuno-compatible pluripotent cell line can be generated without somatic cell transfer.

IPS cells can produce all of the cells of the body due to pluripotency, and unlimitedly produce self-like cells due to self-renewal ability. Human embryonic stem cells also have pluripotency, but are studied and applied in a limited range due to moral issues. However, iPS cells can use human somatic cells, and thus are free from moral issues.

IPS cells are expected to basically treat incurable diseases that are difficult to treat with a drug or surgery by replacing future damaged cells, tissues or organs because of their pluripotency, and moreover, to be applied to various fields of life science including development of a new medicine, and studies of a disease mechanism and embryology. However, iPS cell technology remains in an early stage, and mainly induces dedifferentiation, and therefore focuses on the development of producing and establishing iPS cells. Also, until now, a technique of preparing an animal model in which a human immune system is implemented using human iPS cells and selecting a personalized therapeutic method or drug using such an animal has not been reported.

Conventionally, as a method of establishing a humanized animal model having an immune system similar to a human immune system, a method of grafting hematopoietic stem cells in an immunodeficient mouse was generally used (Greiner D L, Hesselton R A and Shultz L D, Stem Cells 1998; 16: 166-177). However, most of the small amount of human cells produced in the xenografted mouse were B cells, but there were no T cells. Particularly, a humanized mouse established by human hematopoietic stem cells which were xenografted in a conventional immunodeficient mouse had a difference from a normal human body, and particularly, there were obviously limitations to studying the occurrence of a disease or therapeutic effect by stimulation of the immune system.

### DISCLOSURE

### Technical Problem

Therefore, the inventors developed an animal expressing an immune system that is almost the same or the same as a human immune system by injecting previously produced iPS cells into an animal embryo, and completed a technique of more easily and effectively producing a humanized animal. In further detail, in the present invention, iPS cells were produced, and an animal in which a human immune system is expressed using the cells was produced. The animal produced as described above is used to more precisely estimate a human response to a disease inducing material or treating material, and also improves a therapeutic effect.

### Technical Solution

The present invention is directed to providing a method of producing a humanized immune animal using iPS cells and a humanized immune animal produced thereby.

In further detail, one aspect of the present invention provides a method of preparing an animal in which a human immune system is expressed by producing iPS cells by introducing Oct4, Sox2, Klf4 and c-myc genes in human-derived cells; injecting the iPS cells into an embryo of an immunodeficient animal; and implanting the embryo in a uterus.

Another aspect of the present invention provides an animal in which a human immune system is expressed, which is produced by the above-described method.

### Advantageous Effects

The present invention provides a method of preparing an animal in which a human immune system is expressed using rheumatoid arthritis (RA) patient- and osteoarthritis (OA) patient-derived iPS cells, and an animal produced by the above-described method, which can provide a therapeutic agent more suitable for a patient using the animal in which an immune system of the arthritis patient is implemented, and contribute greatly to arthritis treatment.

### DESCRIPTION OF DRAWINGS

FIG. 1 shows synovial cells obtained from an osteoarthritis (OA) patient and a rheumatoid arthritis (RA) patient.

FIG. 2 shows iPS cells produced from the synovial cells of the rheumatoid arthritis (RA) patient.

FIG. 3 shows iPS cells produced from the synovial cells of the osteoarthritis (OA) patient.

FIG. 4 shows stem cell characteristics of the iPS cells produced from the synovial cells obtained from an osteoarthritis (OA) patient and a rheumatoid arthritis (RA) patient, which are analyzed through real-time PCR (qRT-PCR).

FIG. 5 shows stem cell characteristics of the iPS cells produced from the synovial cells obtained from a rheumatoid arthritis (RA) patient through cell staining, compared to a positive control, H7.

FIG. 6 shows stem cell characteristics of the iPS cells produced from the synovial cells obtained from an osteoarthritis (OA) patient through cell staining, compared to a positive control, H7.

FIG. 7 shows the state of a chromosome of the iPS cells produced from the synovial cells obtained from a rheumatoid arthritis (RA) patient, in which both of the shape and number are normal.

FIG. 8 shows a teratoma formed using the iPS cells produced from the synovial cells obtained from a rheumatoid arthritis (RA) patient.

FIG. 9 is a cell staining result showing that a mouse expressing human immune cells is generated.

### EMBODIMENTS OF THE INVENTION

In one aspect of the present invention, the present invention provides a method of preparing an animal in which a human immune system is expressed, which includes: (a) producing iPS cells by introducing Oct4, Sox2, Klf4 and c-myc genes into human-derived cells; (b) injecting the iPS cells into an embryo of an immunodeficient animal; and (c) implanting the embryo in a uterus. Preferably, as the human-derived cells, all cells derived from a human may be used, and cells including somatic cells and reproductive cells and derived from all types of tissues and blood are included.

Such human-derived cells include synovial cells, skin cells, peripheral blood mononuclear cells, fibroblasts, fibrocytes, nerve cells, epithelial cells, keratinocytes, hemocytoblasts, melanocytes, cartilage cells, macrophages, myocytes, hemocytes, marrow cells, lymphocytes (B lymphocytes, T lymphocytes), mononuclear cells, lung cells, pancreatic cells, liver cells, stomach cells, intestinal cells, heart cells, bladder cells, kidney cells, urethral cells, embryonic germ cells, cumulus cells, etc., but the present invention is not limited thereto. Preferably, the human-derived cells are synovial cells, skin cells, peripheral blood mononuclear cells or fibroblasts. In an example of the present invention, as the human-derived cells, synovial cells were used.

The term “synovial cells” used herein are cells of the synovium that covers an inner surface of the glenoid cavity, which are connective tissue cells and classified into two types, for example, A type and B type. A-type cells have a microphage shape, function in phagocytosis, and include a large Golgi apparatus, many lysosomes, and less rough endoplasmic reticula in the cytoplasm. B-type cells are fibroblasts, a surface of which is relatively planar, and in which many rough endoplasmic reticula are in the cytoplasm. The synovium is a tissue covering a joint, and produces a joint fluid. Arthritis has symptoms such as cellular infiltration, edema, and amplification of connective tissues on the synovium.

In the present invention, iPS cells were produced by a method of introducing Oct4, Sox2, Klf4 and c-myc genes into synovial cells.

The term “dedifferentiation (or reprogramming)” used herein refers to a process which can revert to or convert into a final state with a new type of differentiation potential from cells differentiated in different aspects such as cells without differentiation potency or cells with a predetermined part of differentiation potency. In the present invention, a dedifferentiation mechanism may include all of processes of reverting the differentiated cells with a differentiation potency of 0% to less than 100% into a non-differentiated state, and preferably, refers to reversion or conversion of cells partially differentiated with a differentiation potency of more than 0% to less than 100% into cells with a differentiation potency of 100%.

In the present invention, as a dedifferentiation inducer, Oct4, Sox2, Klf4 and c-myc genes were introduced to induce dedifferentiation. The “dedifferentiation inducer” is a material for inducing completely or partially differentiated cells to be iPS cells with a potential to differentiate into a new type. Any material for inducing the dedifferentiation of differentiated cells may be included without limitation, and the dedifferentiation inducing material may be selected depending on the type of cells that will be finally differentiated into, and thus is not limited to Oct4, Sox2, Klf4 and c-myc, which are mentioned above.

The term “iPS cells” are cells induced by artificially performing a dedifferentiation process on completely-differentiated somatic cells, and have pluripotency. In the present invention, iPS cells were produced by introducing Oct4, Sox2, Klf4 and c-myc genes as dedifferentiation inducers, and in Example 2, it is confirmed that the patient-derived iPS cells produced as described above had stem cell characteristics, and iPS cells that can differentiate into various parts were generated.

The present invention provides a method of preparing an animal in which a human immune system is expressed by injecting the patient-derived iPS cells produced by the above-described method into an embryo of an immunodeficient animal, and obtaining an offspring by implantation of the embryo in the uterus of the animal.

The term “immunodeficient animal” used herein refers to an animal which has a decrease or deficiency in an immune response ability due to various causes, for example, a decrease in, detect or dysfunctioning of T cells, B cells, macrophages, antibodies or complements, which are involved in the immune response.

Preferably, the immunodeficient animal is an animal which is deficient in at least one selected from T cells, B cells and natural killer (NK) cells.

Preferably, the immunodeficient animal of the present invention is a severe combined immunodeficient syndrome (SCID) mouse, an SCID-beige mouse, a NOD/Shi-scid/IL-2Rγnull (NOG) mouse, or an NOD scid gamma (NSG) mouse.

The SCID mouse refers to a mutant mouse showing a phenotype of SCID, which is a congenital disorder in a lymphocyte-based stem cell, resulting in deficiencies in all of cellular immunity and humoral immunity due to congenital defects of both lines such as T cells and B cells. The SCID mouse has an autosomal recessive genotype, and defects of functional T cells or B cells due to the dysfunctioning of a recombinase involved in rearrangement of genes for an immunoglobulin or T cell receptor, or a related factor thereof.

Particularly, the SCID-beige mouse has autosomal recessive mutations in both of SCID and Beige. The SCID mutation has the defects of T cells and B cells as described above, and the Beige mutation has the deficiency in NK cells. The SCID-beige mouse shows the both characteristics at the same time, and in other words, has deficiencies in T cells, B cells and NK cells.

The NOG mouse refers to an immunodeficient mouse developed as a receptor for xenografting, which shows double-homozygosity for the SCID mutation and an interleukin-2R (IL-2Rγ) allele mutation, and therefore is deficient in NK cells as well as T cells and B cells.

The NSG mouse is made by mating of a SCID mouse and a non-obese diabetic (NOD) mouse, has neither mature T nor B cells, shows very low NK cell activity, and thus has high efficiency of grafting to human cells.

The method of the present invention is to express a human immune system by injecting iPS cells into an immunodeficient animal. Therefore, any immunodeficient animal as well as the SCID mouse, the SCID-beige mouse, the NOG mouse and the NSG mouse, can be used without limitation.

The term “embryo” used herein refers to an early stage of genesis including a period from one or more cell divisions of a zygote formed by combining sperms and an egg through fertilization to becoming one complete individual. Preferably, the embryo may be a blastocyst stage.

In Example 3 of the present invention, a mouse expressing a human immune system is produced by injecting the patient-derived iPS cells produced in Example 1 into the embryo of an immune cell-deficient SCID beige mouse, and cells showing the same level of staining as human immune cells were detected by an immunocytochemistry (ICC) assay, which will be described in Example 4, thereby observing that the mouse expressing a human immune system is produced.

More preferably, the method of the present invention may further include, before the injection of the iPS cells, treating the mouse with human menopausal gonadotropin (hMG) and human chorionic gonadotropin (hCG).

The HMG includes both active components such as follicle stimulating hormone (FSH) and leuteinizing hormone (LH). These are glycoprotein hormones generated in the pituitary gland, which are used to stimulate the generation of follicles and ovarian growth. The human menopausal gonadotropin (hMG) in the present invention may include a series of hormones such as the human menopausal gonadotropin (hMG) and the leuteinizing hormone (LH), or mutants thereof. The human menopausal gonadotropin (hMG) is secreted from the anterior pituitary in natural circumstances, and may be obtained through extraction or a recombination technique.

Human chorionic gonadotropin (hCG) includes chorionic gonadotropin and thyroid stimulating hormone (TSH), and is synthesized and secreted by the pituitary gland. The human chorionic gonadotropin (hCG) is used to stimulate the ovarian growth, secreted from the anterior pituitary, and may be obtained by extraction or a recombination technique. The human menopausal gonadotropin (hMG) and the human chorionic gonadotropin (hCG) are used to more safely implant the embryo in the uterus. As well as the hormone, any material for secreting the secretion of the hormone, or any material capable of inducing the embryo to be easily implanted in the uterus such as any drug or formula having the effect of the hormone may be used without limitation.

In another aspect of the present invention, the present invention provides an animal in which a human immune system is expressed produced by the above-described method. Preferably, the animal is a mouse.

In the present invention, a ‘humanized mouse’ expressing an immune system the same as or very similar to a human immune system by injecting iPS cells into the immunodeficient mouse was produced, and thus a mouse that can be interpreted to represent a human reaction to a specific material or stimulus was produced. More specifically, the present invention provides a mouse in which an immune system of an arthritis patient is implemented by producing a mouse expressing a human immune system by injecting rheumatoid arthritis patient- and osteoarthritis patient-derived iPS cells, and thus a therapeutic agent suitable for a patient can be developed. Also, when cells of a patient were used as donors, they can enable a study on reactivity to a specific disease or the function of a specific organ possible, may be used as a tool for observing the aftereffects of a drug or the reactivity to a chemical, and furthermore, a treating method and a therapeutic drug, which are suitable for each individual, may be selected through such a model. Particularly, when a process of substituting an immune system as that of each individual is successful according to the present invention, the present invention has a very large useful value in that the application field is expected to expand to medicine, clinical medicine and basic research.

Hereinafter, the present invention will be described in detail with reference to examples. However, the following examples are merely provided to explain the present invention, and the present invention is not limited to the following examples

### EXAMPLE 1

### Produce of iPS Cells

1-1. Patient Gathering and Preparation of Synovia

Rheumatoid arthritis (RA) patients (n=2) and osteoarthritis (OA) patients (n=2), who were diagnosed by classification criteria revised by the American College of Rheumatology (ACR; formerly the American Rheumatism Association) in 1987, were chosen in the inpatient clinic of Rheumatology of Seoul St. Mary's hospital, and synovia was extracted from a total of four patients (two for each group). Synovia samples were obtained from patients undergoing arthroscopic synovectomy or total knee transplant through the surgery. It was determined that, as the patients with osteoarthritis (OA), only people who received early knee osteoarthritis (OA) diagnosis based on the ACR classification criteria were included, and total experiment protocols progressed after receiving an approval by the human research ethics committee of the Catholic University of Korea.

1-2. Separation and Maintenance of RA and OA Synovial Cells

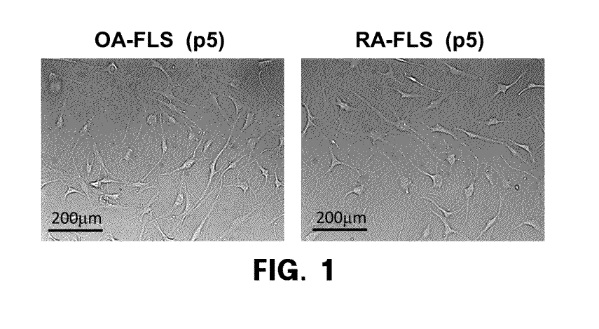

Rheumatoid arthritis (RA) patient- and osteoarthritis (OA) patient-derived synovia were stored in the Sample Bank of the Rheumatoid Research Center before use. Synovial tissues were homogenized, suspended in 0.01% collagenase-containing Dulbecco's modified Eagle's medium (DMEM, Gibco by Invitrogen, Carlsbad, Calif., USA), and mixed for 4 hours at 37° C. Cells were washed, and cultured in DMEM containing 20% fetal bovine serum (FBS) (Gibco by Invitrogen, Carlsbad, Calif., USA) and a 1% penicillin/streptomycin solution (Gibco by Invitrogen, Carlsbad, Calif., USA). FIG. 1 shows synovial cells obtained from rheumatoid arthritis (RA) patient- and osteoarthritis (OA) patient-derived synovia.

1-3. Production of Lentivirus and Transfection of Synovial Cells

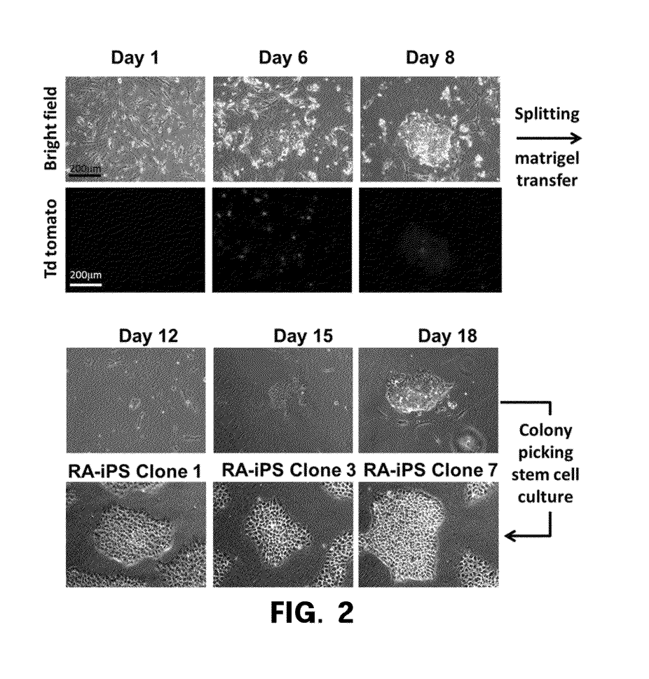

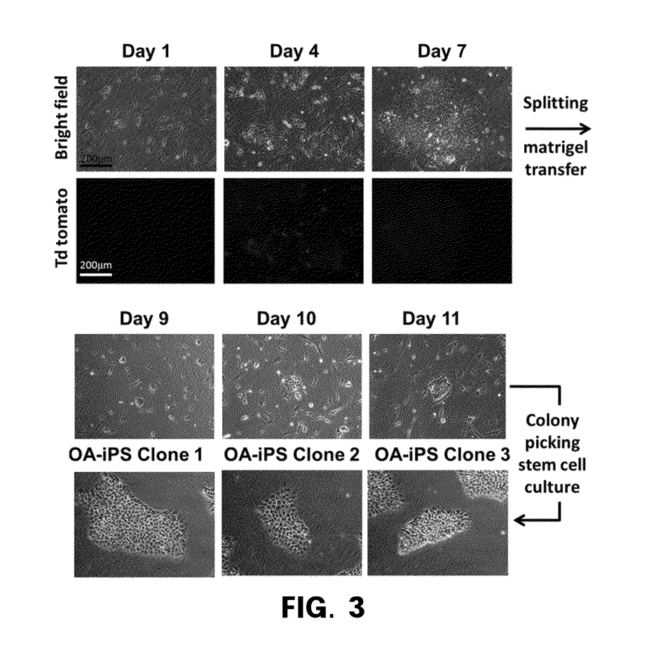

12 mg of 4-in-1 reprogramming plasmids (Oct4, Sox2, Klf4, and c-Myc), 9 mg of packaging pPAX2 plasmids and 3 mg of pMD2G plasmids were transduced into 293T cells (Invitrogen, Carlsbad, Calif., USA) using Lipofectamine 2000 (Invitrogen, Carlsbad, Calif., USA), and then cells were plated to 80% of the surface area of a 100-mm dish. The cells were cultured for about 48 to 72 hours, thereby obtaining a virus, and then the virus was mixed with a Lenti-X Concentrator (Clontech Laboratories, Mountain View, Calif., USA). The resultant mixture was cultured overnight at 4° C., and centrifuged at 1,500 rpm to obtain a viral pellet, and then the viral pellet was resuspended in phosphate buffer saline (PBS). Prior to the transfection of cells with the viruses, first, rheumatoid arthritis (RA) and osteoarthritis (OA) synovial cells were plated on a 6-well plate, and cultured overnight in lentivirus-added media. Afterward, colonies of the generated iPS cells were separated 18 to 20 days after the transfection (FIGS. 2 and 3).

1-4. Culture and Maintenance of Patient-Derived iPS Cells

The rheumatoid arthritis (RA) and osteoarthritis (OA) synovial cells transfected with lentiviruses in Example 3 were cultured in 20% fetal bovine serum (FBS; Gibco by Invitrogen, Carlsbad, Calif., USA)-containing DMEM at 37° C. in 5% CO₂, using all 8 passages. Afterward, the generated patient-derived iPS cells were cultured in a Matrigel-coated culture dish (BD Biosciences, San Jose, Calif., USA) with media for E8 human embryonic stem cells (hESC).

### EXAMPLE 2

### Identification of icP Cells

2-1. Quantitative PCR (qPCR)

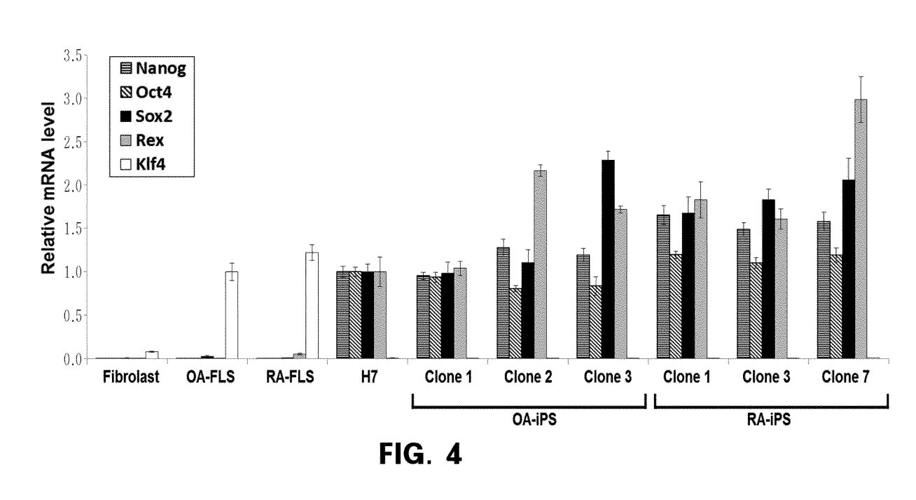

RNA was separated using an RNeasy Plus Mini Kit (Qiagen, Valencia, Calif., USA), and reverse transcriptase PCR (RT-PCR) was carried out using an iScript™ cDNA Synthesis Kit (BIORAD, Marnes-La-Coquette, France). Gene expression was detected by SYBR Green real-time PCR using an ABI Prism 7300 Sequence Detection System (Applied Biosystems, Foster City, Calif., USA). A relative mRNA level was standardized to the GAPDH mRNA level. Therefore, it was determined that the iPS cells produced in Example 1 have stem cell characteristics (FIG. 4).

2-2. Immunostaining of Cells

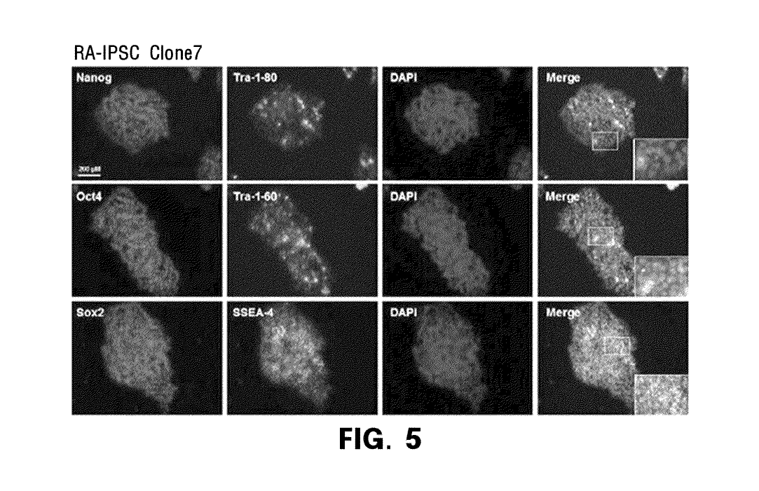

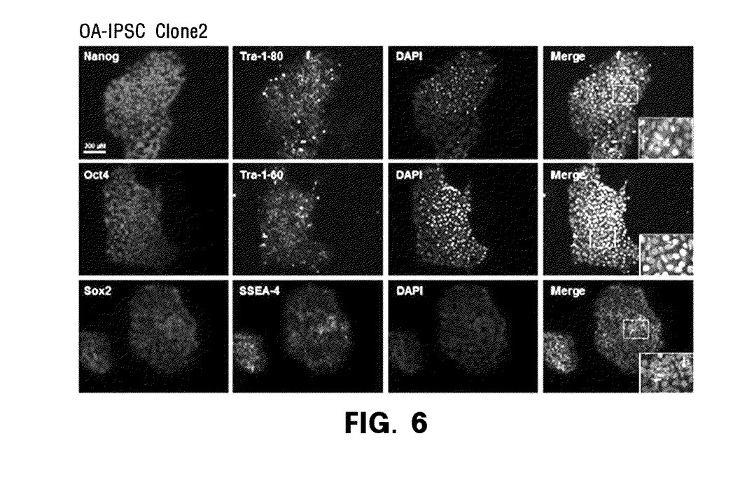

Clones of iPS cells were immobilized in 4% paraformaldehyde, and reacted with SSEA-4, Tra-1-60, Tra-1-80 (Millipore, Billerica, Mass., USA), Oct3/4, Nanog (Santa Cruz Biotechnology, Santa Cruz, Calif., USA) and Sox2 (BioLegend, San Diego, Calif., USA) as primary antibodies for immunostaining. Afterward, Alexa Fluor 594 or 488-binding secondary antibodies (Invitrogen, Carlsbad, Calif., USA) were attached to the primary antibody-attached samples, and observed through an indirect immunofluorescence assay. It was confirmed that each of the rheumatoid arthritis (RA) patient- and osteoarthritis (OA) patient-derived iPS cells produced in Example 2 has stem cell characteristics through the cell staining and the indirect immunofluorescence assay, and particularly, has stem cell characteristics, which are the same as or more than the positive control, H7 (FIGS. 5 and 6).

2-3. Observation of Forming Teratoma

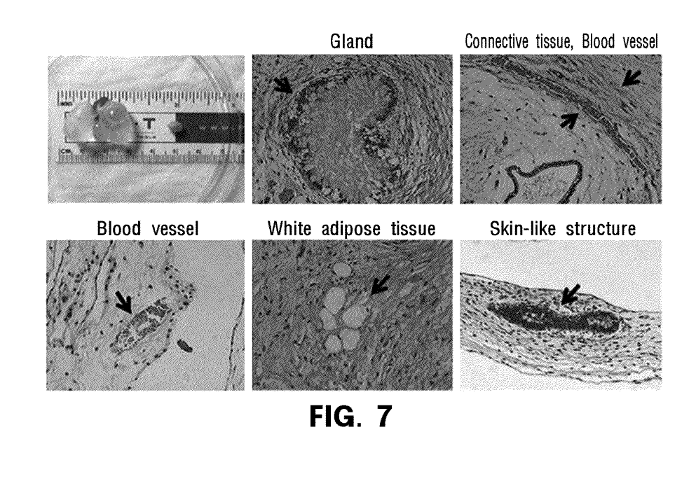

1×10⁶ of the iPS cells generated and maintained according to Example 1 were suspended in 10 mL Matrigel (BD Biosciences, San Jose, Calif., USA). The cells were injected into a capsule in the kidney of an 8-week-old SCID Beige mouse using a 28.5 gauge syringe, and 8 weeks later, generated tumors were extracted, and then underwent hematoxylin and eosin staining. Through the above-described experiment, it was confirmed that the iPS cells having a stem cell potency capable of differentiating into various parts are generated (FIG. 7).

2-4. Karyotype Analysis

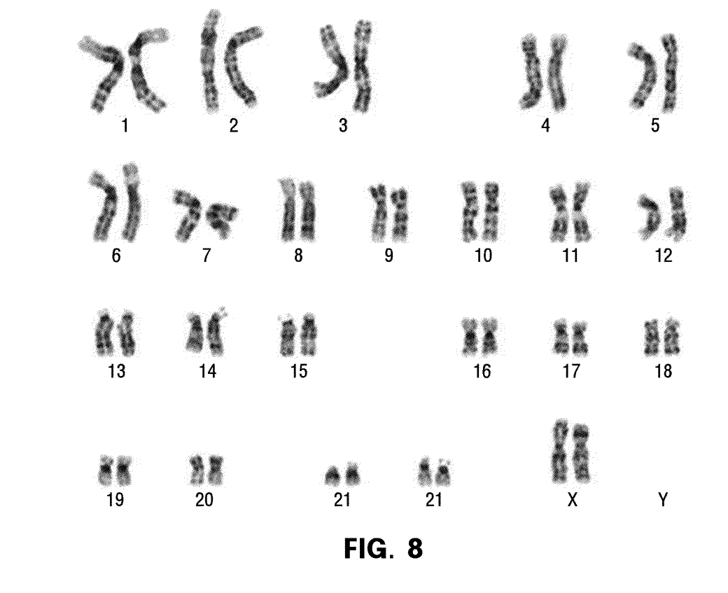

To confirm the characteristics of rheumatoid arthritis patient-derived iPS cells, 30 μL of a chromosome resolution additive (CRA; Genial Genetic Solutions Limited, The Heath Business & Technical Park, Runcorn, U.K.) was put into a 6-well plate, and the cells were cultured for 1 hour and treated with colcemid for 30 minutes to stop cell division. The cells were separated using trypsin, and treated with a pre-heated KCl hypotonic solution. The cells were immobilized with a solution prepared by mixing acetic acid and methanol in a ratio of 1:3 and attached to a slide, and then karyotype analysis was carried out by Trypsin-Giemsa banding. Therefore, it was confirmed that the shape and number of chromosomes of the rheumatoid arthritis patient-derived iPS cells were all normal, and there was no mutation (FIG. 8).

### EXAMPLE 3

### Production of Mouse Expressing Human Immune System

To obtain a mouse in which an immune system of an arthritis patient is implemented, a patient-derived stem cell-injected embryo was implanted in the uterus of a mouse. In this experiment, 10-week-old male and 6-week-old female CD-1 strains were used, and mice that would undergo operations before the experiment were treated with human menopausal gonadotropin (hMG) and human chorionic gonadotropin (hCG) at 50 IU/ml each to have a concentration of 0.1 ml/mouse. Patient-derived iPS cells were detached with 1 mg/ml accutase, and 5 to 8 of the iPS cells were injected into an embryo. 37 of the cell-injected embryos were implanted into two pseudo-pregnant female mice, and about three weeks later, the mice produced 9 offspring.

### EXAMPLE 4

### Confirmation of Expression of Human Immune System

A human immune system-expressed mouse was produced by injecting patient-derived iPS cells into an immune cell-deficient SCID beige mouse, and identified by an immunocytochemistry (ICC) assay. For the ICC assay, blood was taken from the mouse, spread on a slide to dry at room temperature for 1 hour, immobilized with acetone for 10 minutes, and then dried at room temperature. Afterward, the resultant product was washed with PBST buffer (washing buffer), and blocked with 10% normal goat serum at room temperature for 1 hour. After then, a monoclonal rabbit anti-CD3E antibody (abcam) as a primary antibody was diluted in a ratio of 1:100 and applied to the slide, and allowed to react overnight at 4° C. After the reaction, the slide was immersed again in PBST buffer (washing buffer) to wash, and a biotinylated secondary goat anti-rabbit IgG as a secondary antibody was diluted in a ratio of 1:200 and applied to the slide, and allowed to react for 1 hour at room temperature. After the reaction, the slide was immersed again in PBST buffer (washing buffer) to wash, cultured in 0.6% H₂O₂ (SAMCHUN Catalog #7722-84-1) for 10 minutes, and then washed again with PBST buffer for 5 minutes. Two drops each of streptavidin-HRP and ready-to-use (R.T.U.) reagents (Vector Lab. Catalog #SA-5704) were dropped on the slide to allow to react for 1 hour at room temperature, and washed with PBST buffer for 5 minutes. Color development was checked using a DAB peroxidase substrate kit (Vector Lab Catalog #SK-4100) at room temperature, and the reaction was stopped at a desired degree of the color development with tap water. Afterward, the dried slide was immersed in xylene to wash, and mounted with a mounting medium (Vector Lab Catalog #H-5000).

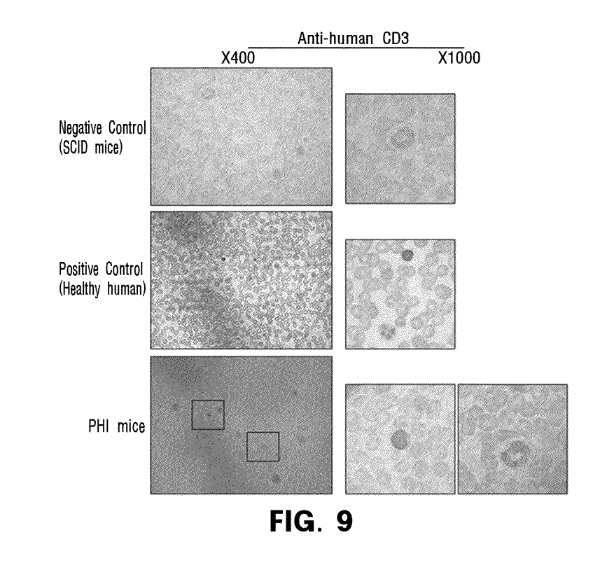

Through the staining, it was confirmed that no cells positive in the staining were found in the negative control, and cells having the same shape and staining level as those of human immune cells, which is the positive control, were shown in a human immune system-expressed mouse (FIG. 9). Therefore, it was confirmed that the humanized mouse expressing human immune cells was normally produced.

1. A method of preparing an animal in which a human immune system is expressed, comprising:
(a) preparing iPS cells by introducing Oct4, Sox2, Klf4 and c-myc genes to human-derived cells;
(b) injecting the iPS cells into an embryo of an immunodeficient animal; and
(c) implanting the embryo in a uterus.

2. The method of claim 1, wherein the human-derived cells are somatic cells or reproductive cells.

3. The method of claim 1, further comprising:
after the (a) operation and before the (b) operation,
treating the mouse with human menopausal gonadotropin (hMG) and human chorionic gonadotropin (hCG).

4. The method of claim 1, wherein the immunodeficient animal is deficient in at least one selected from the group consisting of T cells, B cells and natural killer (NK) cells.

5. The method of claim 4, wherein the immunodeficient animal is a severe combined immunodeficient syndrome (SCID) mouse, an SCID-beige mouse, a NOD/Shi-scid/IL-2Rγnull (NOG) mouse, or an NOD scid gamma (NSG) mouse.

6. An animal in which a human immune system is expressed, which is produced by claim 1.

7. The animal of claim 6, wherein the animal is a rodent.

8. The animal of claim 6, wherein the animal consists of at least one human-derived cells selected from the group consisting of T cells, B cells and NK cells.
